(19) United States
(12) Patent Application Publication
(10) Pub. No.: US 2015/0167093 A1
(43) Pub. Date: Jun. 18, 2015

(54) METHOD FOR IDENTIFYING INCREASED RISK OF ANXIETY DISORDERS

(71) Applicant: CORNELL UNIVERSITY, Ithaca, NY (US)

(72) Inventors: Francis S. LEE, New York, NY (US); BJ CASEY, New York, NY (US)

(73) Assignee: CORNELL UNIVERSITY, Ithaca, NY (US)

(21) Appl. No.: 14/633,634

(22) Filed: Feb. 27, 2015

Related U.S. Application Data
(63) Continuation of application No. 13/522,079, filed on Sep. 5, 2012, now abandoned.
(60) Provisional application No. 61/295,090, filed on Jan. 14, 2010.

(51) Int. Cl. C12Q 1/68; A61B 5/00; A61B 5/16; G06F 19/00; A61B 5/055

(52) U.S. Cl. CPC C12Q 1/6883; G06F 19/345; A61B 5/055; A61B 5/165; A61B 5/7275; C12Q 2600/156

(57) ABSTRACT

The present invention is for methods for identifying a human having an increased risk for anxiety disorders. The method involves genotyping the human for a specific brain-derived neurotrophic factor (BDNF) single nucleotide polymorphism (SNP), and/or administering a fear conditioning procedure while measuring fear, and administering an extinction procedure while measuring fear. The method can also involve comparing/MRI images of the amygdala of the human acquired during conditioning and extinction and determining if the human is unresponsive to extinction therapy by noting heightened and non-declining activity in the amygdala during extinction.

### CROSS-REFERENCE TO RELATED APPLICATION

This application claims the benefit of U.S. Provisional Application No. 61/295,090, filed Jan. 14, 2010, which is incorporated herein by reference in its entirety.

This invention was made with Government support under Grant Number MH079513, MH060478, NS052819, HD055177, and GM07739 awarded by the National Institutes of Health. The United States Government has certain rights in the invention.

### BACKGROUND OF THE INVENTION

Genetically modified mice provide useful model systems for testing the role of candidate genes in behavior. The extent to which such genetic manipulations in the mouse and the resulting phenotype can be translated across species, from mouse to human, is less clear.

Brain-derived neurotrophic factor (BDNF) mediates synaptic plasticity associated with learning and memory, specifically, in fear learning and extinction. BDNF-dependent forms of fear learning have known biological substrates, and lie at the core of a number of clinical disorders associated with variant BDNF.

Fear learning paradigms require the ability to recognize and remember cues that signal safety or threat and to extinguish these associations when they no longer exist. These abilities are impaired in anxiety disorders such as post-traumatic stress disorder and phobias. Behavioral treatments for these disorders, such as exposure therapy, rely on basic principles of extinction learning in which an individual is repeatedly exposed to an event that was previously associated with aversive consequences.

Understanding the effect of variations of the BDNF allele on these forms of learning can provide insight into the mechanism of risk for anxiety disorders, refine existing treatments, and may lead to genotype-based personalized medicine.

### SUMMARY OF THE INVENTION

In an embodiment of the invention, a method for identifying an increased risk for anxiety disorders in a human is provided. The method comprises testing the human for the presence of brain derived neurotrophic factor (BDNF) Val-66-Met genotype, and identifying an increased risk for anxiety disorders in humans with the BDNF Val-66-Met genotype.

In one embodiment of the invention, the anxiety disorder is post traumatic stress disorder. In another embodiment, the anxiety disorder is a phobia.

### BRIEF DESCRIPTION OF THE FIGURES

FIG. 1. Altered extinction in mice and humans with BDNF Val66Met.

Impaired extinction in Met allele carriers (Val/Met and Met/Met) as a function of time in 68 mice (A) and 72 humans (B) as indexed by percent time freezing in mice and skin conductance response (SCR) in humans to the conditioned stimulus when it was no longer paired with the aversive stimulus. All results are presented as a mean+SEM. *p<0.01, Student's t test. =p<0.02, Student's t test. VV=Val/Val; VM=Val/Met; MM=Met/Met.

FIG. 2. Impaired learning of neutral cue in human Met allele carriers.

Elevated skin conductance response (SCR) to the cue never paired with the aversive stimulus during fear conditioning as a function of time in Met allele carriers (VM) relative to non-Met allele carriers (VV). All results are presented as a mean±SEM. *p<0.001, Student's t test. VV=VallVal; VM=Val/Met.

FIG. 3. Neural circuitry of the behavioral effect of BDNF Val66Met during extinction.

(A) Average percent freezing during extinction by genotype in 68 mice.

(B) Average skin conductance response (SCR) during extinction by genotype in 72 humans.

(C) Brain activity as indexed by percent change in MR signal during extinction in the ventromedial prefrontal cortex (vmPFC) by genotype (xyz=−4, 24, 3), with Met allele carriers having significantly less activity than Val/Val homozygotes [VM<VV=blue], image threshold p<0.05, corrected. (D) Genotypic differences in left amygdala activity during extinction (xyz=−25, 2, −20) in 70 humans, with Met allele carriers having significantly greater activity than Val/Val homozygotes [VM>VV=orange], image threshold p<0.05, corrected. *p<0.05. **MM were included in the analysis with VM, but plotted separately to see dose response. All results are presented as a mean+SEM. VV ValNal; VM=Val/Met; MM=Met/Met; MR=magnetic resonance.

FIG. 4: Conditioning in mice and humans with BDNF Val66Met.

BDNF genotyoe did not affect fear conditioning in 65 mice (A) and 72 humans (B) as indexed by percent time freezing and skin conductance (SCR) respectively. Genotype did not affect the amount of time mice spent freezing during conditioning. Human non-Met allele carriers (Val/Val) and Met allele carriers Val/Met) differentiated between the CS+ (cue paired with aversive stimulus) and CS− safety cue), while the interaction between genotype and CS type was not significant [F(1,70)=0.67, p<0.42]. Therefore, any observed extinction effects are not due to impaired initial learning of the contingencies. All results are represented as mean±SEM. *p<0.001, paired t-test. NS+ not significant; VV=Val/Val; VM=Val/Met; MM=Met/Met.

FIG. 5: Percent time freezing during extinction ITI.

Line graph depicting the average percent time freezing for BDNF^{met/met} (n=4) and BDNF^{val/val} (n=4) mice during the intertribal interval (ITI) over the course of fear extinction learning. The average percent time freezing is presented for each of the first 10 ITI's and demonstrate no significant difference in the present time freezing between genotypes.

FIG. 6: Whole brain DTI analysis shows non-Met allele carriers have a higher fractional anisotrophy in the uncinate fasciculus than Met allele carriers.

(A) The left (xyz=−20, 20, −6) and right (xyz=27, 16, −5) uncinate fasciculus shown on an axial slice (p<0.05, corrected).

(B) Val/Val homozygotes have higher FA values in the uncinate fasciculus compared to Met allele carriers. Met/Met homozygotes were included in the analysis with Val/Met subjects, but plotted separately to see dose response. VV=Val/Val; VM=Val/Met; MM=Met/Met.

FIG. 7: Association between structure and function in the prefrontal cortex.

Activity in the vmPFC during extinction was correlated with fractional anisotropy in the uncinate fasciculus in Val/Val homozygotes (r=0.47, p<0.01) but not in Met allele carriers (r+0/24, p<0.17). VV=Val/Val; VM=Val/Met.

FIG. 8: Tables 1 and 2: Demographics of skin conductance and participants with fMRI, respectively

### DETAILED DESCRIPTION OF THE INVENTION

The present invention focuses on identifying biologically valid phenotypes across species. A common single nucleotide polymorphism (SNP) in the brain-derived neurotrophic factor (BDNF) gene that leads to a valine (Val) to methionine (Met) substitution at codon 66 (Val66Met) is the subject of this invention. In an inbred genetic knock-in mouse strain that expresses the variant BDNF allele to recapitulate the specific phenotypic properties of the human polymorphism in vivo, it has been discovered that the BDNF Val66Met (hereinafter “Met allele”) genotype is associated with treatment resistant forms of anxiety-like behavior.

One objective of this invention is to demonstrate that the Met allele genotype impacts extinction learning in a mouse model, and that the results can be generalized to human populations.

Another objective of this invention is to demonstrate the impact of the Met allele on classic fear conditioning and extinction paradigms adapted to be suitable for each species and that are associated with well known underlying biological substrates. Fear conditioning consisted of pairing a neutral cue with an aversive stimulus. With repeated pairings, the cue itself takes on properties of the aversive stimulus as it predicts threat of an impending aversive event. Extinction consisted of presenting the cue alone following conditioning, whereby the association is diminished with repeated exposure to empty threat.

As will be discussed below in the Examples, 68 mice were tested (17 BDNF^{Val/Val}, 33 BDNF^{Val/Met} and 18 BDNF^{Met/Met}) and 72 humans group-matched on age, gender and ethnic background (36 Met allele carriers: 31 BDNr^{Val/Met} and 5 BDNF^{Met/Met} and 36 non-Met allele carriers: BDNF^{Val/Val}-Table S1). It was found that there was no effect of the BDNF Met allele on fear conditioning in mice as measured by percent freezing behavior to the conditioned stimulus (F(2,6S)=1.58, P<0.22) (FIG. 4A) or on general fear arousal as measured by freezing during the intertrial interval (ITI) (FIG. 5). We grouped human Met allele carriers together (Val/Met and Met/Met) for analyses because the rarity of human Met allele homozygotes prevents enough observations for meaningful analysis.

Similar to the mouse findings, there was no effect of the BDNF Met allele on fear conditioning in humans as measured by skin conductance response to the cue predicting the aversive stimulus relative to a neutral cue (F(1,70)=0.67, P<0.42) (FIG. 4B).

Analysis of extinction trials showed a main effect of genotype for both mice ((F(2,65)=6.55, p<0.003); Val/Val: 48.8±2.3; Val/Met: 53.2±1.8; Met/Met: 61.3±2.8) and humans ((F(1,70)=4.86, P<0.03); Val/Val: 0.32±0.03; Val/Met: 0.42±0.04), such that extinction learning was impaired in Met allele carriers relative to non-Met allele carriers.

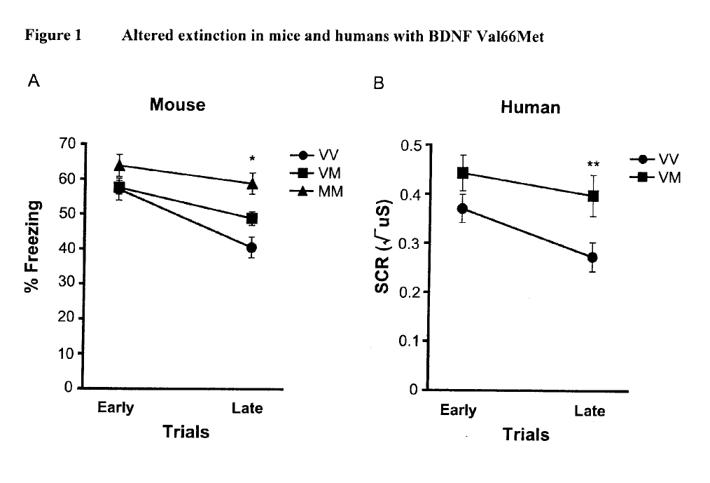

The Met allele carriers showed slower extinguishing as indicated by an interaction of time X genotype for the mouse (F(2,65)=6.51, P<0.003) (FIG. 1) with no differences in freezing initially, but a dose response of the Met allele on percent freezing behavior during late trials (Val/Val vs Val/Met: t(48)=−2.62, P<0.01; Val/Val vs Met/Met: t(33)=−4.78, P<0.0001; Val/Met vs Met/Met: t(49)=−2.90, P<0.006).

Humans showed a similar pattern to the mice with no genotypic difference in the initial human skin conductance response during early trials of extinction (470)=−1.57, p<0.12), but significant differences by late trials (t(70)=−2.43, p<0.02, corrected for time). These data demonstrate slower or impaired extinction related to the Met allele in both mouse and human.

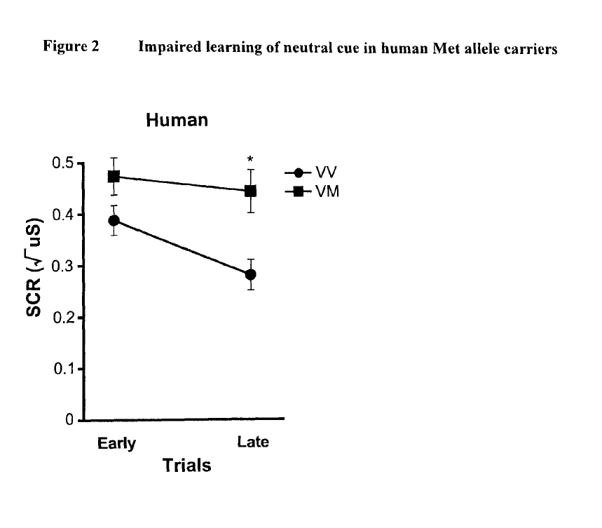

The learning paradigm for humans included a conditioned stimulus paired with the aversive stimulus and a neutral stimulus that was not paired with the aversive stimulus. This design allowed for distinguishing between effects due to impaired learning versus a general effect of heightened anxiety, as generalized heightened anxiety would lead to a similar response to both the conditioned and neutral cues. Met allele carriers had an overall heightened response to both conditioned and neutral cues [main effect of genotype (F (1,70)=7.21, p<0.009)], but overall differentiated between the conditioned and neutral cues similar to the non-Met allele carriers (FIG. 4B). Yet, when examining these effects over time, Met allele carriers took longer to recognize that the neutral cue was not associated with the aversive stimulus, as evidenced by significant genotypic differences during late trials (470)=−3.46, p<0.001, corrected for time) but not early trials (t(70)=−1.44, P<0.16) (FIG. 2). Thus, the skin conductance response to the neutral cue during fear conditioning, showed a similar pattern as that observed during extinction trials.

The genetic findings for both fear conditioning and extinction suggest that learning about cues that signal threat of an impending aversive event is intact in Met allele carriers. However, learning that cues no longer signal threat (e.g., extinction) or do not predict threat (cues not paired with an aversive stimulus) is impaired in Met allele carriers, leading to exaggerated and longer retention of aversive responses where they are not warranted.

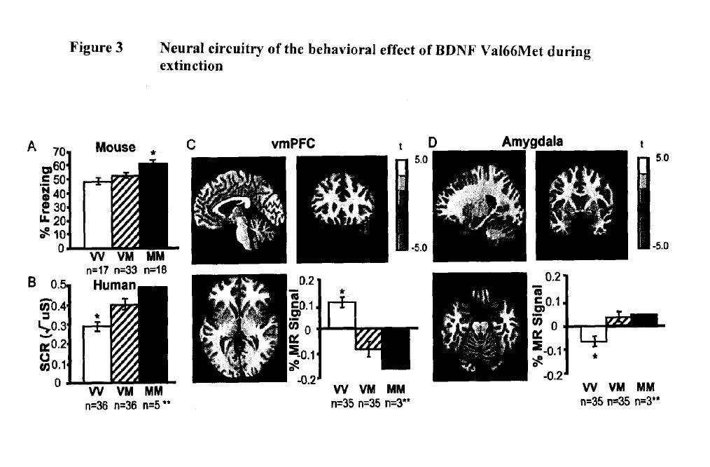

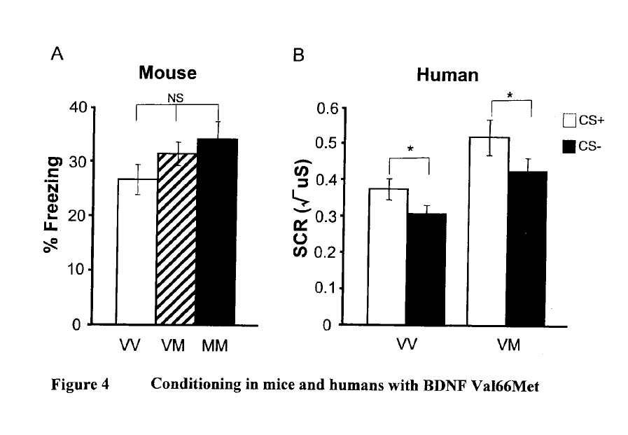

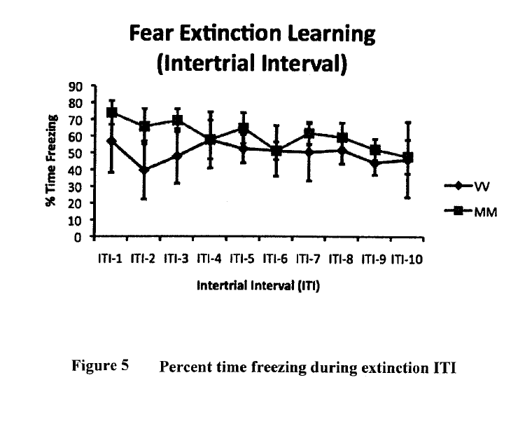

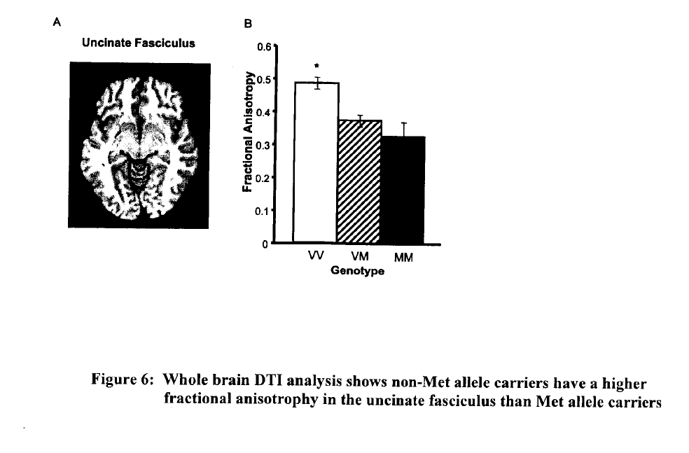

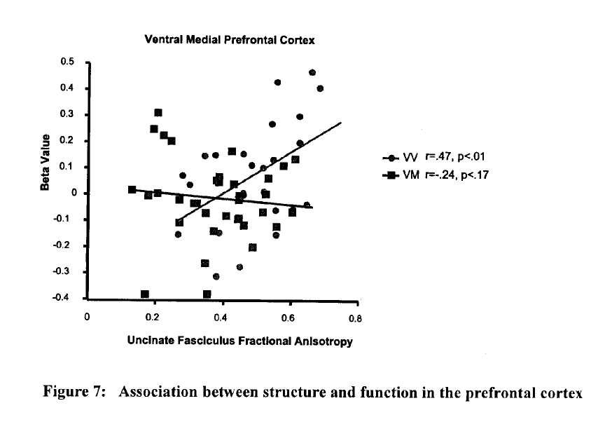

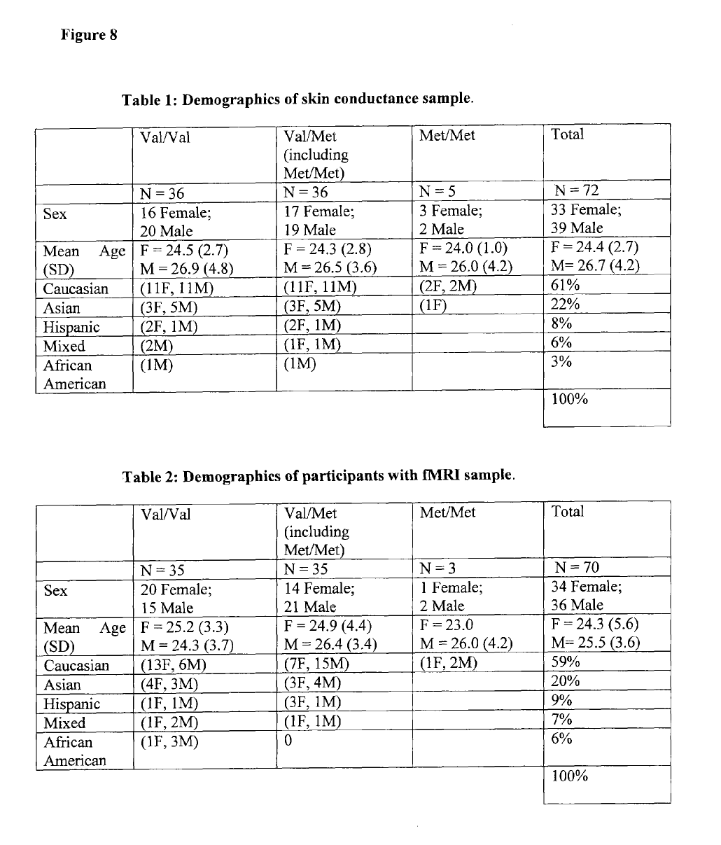

To provide neuro-anatomical evidence to validate cross-species translation, human functional magnetic resonance imaging (fMRI) was used to define the underlying neural circuitry of the behavioral effects of BDNF Val66Met and map them to known circuits involved in fear learning in the rodent (FIG. 3). We targeted frontoamygdala circuitry that has been demonstrated to support fear conditioning and extinction in both rodent and human studies. Whereas portions of the amygdala have been shown to be essential for fear conditioning, ventral prefrontal cortical regions have been shown to be important for modifying previously learned associations and extinction.

Thus, based on the behavioral findings in the mouse and human uncovered in the instant invention, it is believed that ventromedial prefrontal regions, important in extinction, are less active in Met allele carriers relative to non-Met allele carriers and that amygdala activity may be enhanced.

The main effect of genotype on brain activity during extinction of the previously conditioned stimulus was examined. The analysis directly parallels the observed behavioral main effects of genotype on extinction as measured by mean percent time freezing in the mice (FIG. 3A) and mean skin conductance response in humans (FIG. 3B) with Met allele carriers showing weaker extinction. The imaging results showed significantly less ventromedial prefrontal cortical (vmPFC) activity during extinction in Met allele carriers relative to non-Met allele carriers (t(68)=−3.78, p<0.05, corrected), (FIG. 3C). In contrast, Met allele carriers show greater amygdala activity relative to non-Met allele carriers during extinction (t(68)=2.23, P<0.05, corrected) (FIG. 3D). These findings indicate that cortical regions previously shown to be essential for extinction (vmPFC) in both rodent and human are hypo-responsive in Met allele carriers relative to non-Met allele carriers. Moreover, Met allele carriers show continued recruitment of the amygdala, a region that should show diminished activity during the extinction trials of the experiment. These findings are most likely due to the SNP biasing activity-dependent learning rather than affecting CNS development per se, as there was no evidence of genotypic developmental effects on brain structure in this ethnicity-, age- and gender-matched sample using MR1-based brain morphometry.

The herein experiments identify a behavioral phenotype related to BDNP Val66Met across species providing evidence for translation from mouse to human. The mouse model provides the opportunity to test dose-dependent effects of the BDNF Met allele in both a controlled genetic and environmental background not feasible in humans. These features allow for reliable assignment of behavioral differences to the effects of the Val66Met polymorphism.

The human behavioral and imaging findings provide confidence that cross-species translation is biologically valid, by defining the underlying neural circuitry of the behavioral effects of BDNF Val66Met that can be mapped onto known circuits involved in fear learning and extinction. The robustness of our findings across species and paradigms is evidenced by work showing slower extinction coupled with decreased neuronal dendritic complexity in vmPFC in the BDNF^{Met/Met} mice in a conditioned taste aversion task compared with wild-type counterparts.

Impaired extinction learning has been implicated in anxiety disorders, including phobias and post-traumatic stress disorder, whereby the individual has difficulty recognizing an event as safe. Our neuroimaging findings of diminished ventromedial prefrontal activity and elevated amygdala activity during extinction are reminiscent of those reported in patients with anxiety disorders and depression when presented with empty threat or aversive stimuli (e.g., fearful faces).

Understanding the effect of the BDNF Met allele on specific components of a simple form of learning provides insight into risk for anxiety disorders and has important implications for the efficacy of treatments for these disorders that rely on extinction mechanisms.

One such treatment is exposure therapy whereby an individual is repeatedly exposed to a traumatic event in order to diminish the significance of that event. Our findings suggest that the BDNF Val66Met SNP may play a key role in the efficacy of such treatments and may ultimately guide personalized medicine for related clinical disorders.

### Examples

### BDNFMel Mice

A gene-targeted BDNF knock-in mouse containing the genetic variant BDNF (BDNFMct) was created using a targeting vector that replaced the coding region of the BDNF gene with BDNF_{Met} (S1). In this mouse, transcription of BDNF_{Met} is regulated by endogenous BDNF promoters. These BDNF_{Met} mice were backcrossed onto C57/B16 background for at least 10 generations (F10) prior to experimentation. ^{BDNFVal/Met} mice were intercrossed to produce BDNF^{Val/Val}, BDNF^{Val/Met} and BDNF^{Met/Met}.

In order to reduce experimental variability, age-matched littermate pairs resulting from heterozygous crossings were used in this experiment. Adult wild-type (BDNF^{Val/Val}) and littermate heterozygotes (BDNF^{Val/Met}) and BDNF^{Met/Met} male mice that were 2-3 months old in age were used. The Weill Cornell Medical College Institutional Animal Care and Use Committee approved all procedures relating to animal care and treatment. All animals were kept on a 12:12 light-dark cycle at 22° C. with food and water available ad-libitum. All experimental manipulations were performed during the light-on phase of the cycle in accordance with institutional guidelines. All behavioral measurements were performed by raters blind to genotype. A total of 68 mice were tested: 17 BDNF^{Val/Val}, 33 BDNF^{Val/Met} and 18 BDNF^{Met/Met}.

### Fear Conditioning Apparatus

The conditioning apparatus consisted of a standard mouse shock-chamber (Coulbourn Instruments Mouse Test Cage, PA) set up in a sound attenuated box and scented with peppermint odor (0.1% peppermint). The conditioned stimulus (CS) was a 30 s, 70 dB, 5 kHz tone presentation. The unconditioned stimulus (US) was a 0.7 rnA shock delivered through the grid floor. Stimuli presentations were controlled by a PC computer using Graphic State software interfaced to the chamber. Conditioned freezing responses were recorded with video cameras mounted to the top of the conditioning chamber.

### Fear Conditioning and Extinction Procedure

Following a three minute acclimation period to the conditioning chamber, mice received three conditioning trials consisting of a 30 s presentation of a (5 kHz, 70 dB) tone (CS) that coterminated with a 0.7 rnA foot shock (US) during the last 1.0 s of the tone. Each conditioning trial was separated by a 30 s inter-trial interval. Four minutes following the end of conditioning, the extinction procedure began in which mice were exposed to 30 presentations of the CS in the absence of the US. Tone presentations lasted 30 s and were separated by a 30 s intertribal interval. Following extinction, mice were returned to their home cages.

Mice were videotaped during the entire protocol for subsequent quantification of behavior. Freezing responses during training were analyzed using video recordings by raters blind to mouse genotype. Freezing was characterized by a crouching posture in the absence of visible movement except that due to respiration. The time spent freezing during the initial acclimation to the chamber period was measured and served as an assay for unconditioned effects on general activity levels. Freezing behavior was ascertained for each presentation of the CS during both conditioning and extinction. Percent time spent freezing was calculated by dividing the amount of time spent freezing during the 30 s tone presentation by the duration of the tone (30 s) itself.

Extinction trials were binned into early and late trials. Early trials represent the average of the first 15 trials, while late trials represent the average of the last 15 trials. Data were analyzed with repeated measures GLM followed by post-hoc t-tests, where appropriate. Data analyses were performed using SPSS statistical program version 16.0.

The use of immediate extinction in this task parallels the human paradigm described below. Some data suggests that extinction conducted immediately after fear learning may erase or prevent the consolidation of the fear memory trace. However, immediate and delayed extinction both have been shown to share spontaneous recovery and reinstatement in rats and humans. These findings suggest that immediate extinction does not erase the original memory trace, but instead requires new learning that acts to suppress fear expression without erasing the original memory trace, similar to delayed extinction. In addition, our results using immediate extinction replicate our previous study using delayed extinction in a conditioned taste aversion task, in that BDNF^{Met/Met} mice showed impaired extinction.

### Human Participants

Prior to participating in this study, subjects were pre-screened for exclusion criteria, which included left-handedness, hearing impairment, a present or past diagnosis of a psychiatric condition, head trauma or concussion, a first degree relative with a history of a psychiatric condition and any contraindication for MRI (claustrophobia, metallic implants). Prior to participation, all subjects provide informed written consent approved by the Institutional Review Board and were compensated for their participation.

133 right-handed volunteers between the ages of 18 and 35 years completed the fear conditioning and extinction paradigm. SCR was successfully recorded from 111 participants with 13 subjects showing non-measurable levels of skin conductance, and technical/equipment problems with 9 participants. To avoid spurious allelic associations to rs6265, we balanced demographic factors, including age, gender, and ethnicity across genotype categories (FIG. 8, Table 1). We also performed ethnicity-specific analyses and found that the effect of the Met allele on extinction and conditioning, as measured by change in SCR with time, was not driven by any single ethnic group (extinction: F (3,64)=0.32, p<0.81) or (conditioning: F (3,64)=0.69, P<0.56). Functional neuroimaging data were obtained from 104 subjects (34 were discarded due to greater than 3% fluctuation in MR signal throughout scan, head movement greater than 2 mm translation or 2° of rotation on more than 5% of the trials, and/or noncompliance by the participant). There were 70 usable scans representing 35 per genotypic group (Met allele or non-Met allele) (FIG. 8, Table 2). Due to the small number of BDNF^{Met/Met} subjects, Met allele carriers were combined in all analyses of human data but plotted to see dose response of allele.

DNA Collection. Extraction and Analysis

Saliva samples were collected from each subject tested and used as a source of genomic DNA for genetic analysis. Saliva samples (˜4 cc total) were collected and DNA extracted using the Oragene system (DNA Genotek). A Taqman 5′ exonuclease assays (ABI) was used to genotype DNA samples at the BDNF Val66Met (rs6265) SNP. Assays were performed on a 7900HT apparatus (ABI) in real-time PCR mode using standardized cycling parameters for ABI Assays on Demand Allelic. Fluorescence intensities were also collected in Allelic Discrimination mode after thermal cycling. Visual inspection of the amplification curves for each allele of rs6265 led to determination of the genotype. All samples were required to give clear and concordant results in real time and endpoint analyses and all samples that did not were re-run and/or re-extracted until they provided clear genotype calls.

### Stimuli and Experimental Task

Subjects completed nine runs of a fear learning task that was divided into three consecutive phases: fear acquisition, reversal, and extinction. A simple discrimination paradigm with a partial reinforcement schedule was used. Conditioned stimuli were neutral geometric shapes (blue and yellow colored squares). The unconditioned stimulus (US) was white noise combined with a 1000 Hz tone, which was intensity tiered for smooth onset and offset. Sound intensity was measured by an audiometer and presented at 95 dB. The auditory stimulus was generated and modified using the digital audio editor Audacity 1.2.6. The aversiveness of the US was validated previously and its effectiveness in conditioning was confirmed by a significantly greater SCR to the cue predictive of the aversive stimulus relative to the neutral cue, during both the acquisition and reversal phases (F(1,7I)=29.46, p<0.0001).

Trial onset began with cue presentation for 3 s. The US was presented for 1 s and coterminated with one of the conditioned stimuli on 50% of the presentations. This partial reinforcement schedule allowed us to examine the response to conditioned stimuli that predicted the US without being contaminated by response to the US itself. All reported findings involving the CS trial type contained only trials in which the CS was presented in the absence of the US. Separate analysis of the unconditioned stimulus trials showed no genotypic difference ((F(1,70)=1.29, P<0.26); Val/Val: 0.80±0.06; Val/Met: 0.90±0.06). Therefore, our reported genotypic findings are not due to greater reactivity by the Met allele carriers to the aversive stimulus used in the fear conditioning paradigm.

Each trial lasted 16 s with a 13 s inter-trial interval (ITI) during which a fixation cross was presented. Timing of events was based on the hemodynamic response of the blood oxygenation level dependent response, on which the imaging results were based, and time course of the skin conductance response, on which the behavioral measure was based, to ensure decoupling of experimental events for both measures. During acquisition, one square (e.g., blue) was paired with the US on half of the trials (conditioned stimulus), and the other (e.g., yellow) was never paired with the US (neutral stimulus), counterbalanced across participants. During a reversal condition the previous neutral stimulus was paired with the US on half of the trials and the previous conditioned stimulus was not paired with the US. In extinction, both colored squares were presented in the absence of the US.

Stimuli were presented in a pseudorandom order, with the same stimulus not being presented more than twice consecutively and no consecutive reinforced trials. Subjects were not told the objectives of the experiment, but were only informed that they would see different colored geometric shapes and that they would sometimes hear a loud noise. Each run lasted four minutes and 26 s, in which 16 stimuli were presented. Each phase consisted of 3 runs. A total of 24 conditioned stimulus trials, of which half coterminated with the unconditioned stimulus and 24 neutral stimulus trials were presented during both the acquisition and reversal phases. Extinction consisted of presentations of each conditioned stimulus without the US.

### Stimuli and Apparatus

Subjects viewed stimulus images on an overhead liquid crystal display (LCD) panel in the bore of the MR scanner with the Integrated Functional Imaging System-Stand Alone (IFIS-SA; fMRI Devices Corporation, Waukesha, Wis.). E-Prime software (Psychology Software Tools, Inc, Pittsburgh, Pa.) controlled the presentation of visual and auditory stimuli. Auditory stimuli were presented through noise-canceling headphones in the scanner (fMR1 Devices Corporation, Waukesha, Wis.). Foam padding was placed around the head to help reduce motion.

### Physiological Assessment and Analysis

An MRI compatible skin conductance recording system (SCRIOOC Biopac, Goleta, Calif.) together with the AcqKnowledge (Biopac) software was used to amplify and record the skin conductance response (SCR). Eprime software generated TTL timestamps for each stimulus (conditioned stimulus, neutral stimulus, unconditioned stimulus) that were recorded on the Biopac channel recording. SCR was acquired using disposable electrodermal gel electrodes attached to the distal phalanx of the second and third digits of the left hand. The SCR was sampled at a rate of 200 Hz and a 1 Hz filter was applied (Gain 2 μmho/V). SCR waveforms were analyzed using Matlab. Data were smoothed and local peak detection was determined for each individual subject's data. Stimulus related amplitude differences were measured as trough to peak conductance differences occurring within a time window of 1 to 8 s following stimulus onset. The amplitude of the largest SCR associated with each stimulus during this time frame was used as an index of maximum arousal. The raw skin conductance scores were square root transformed to normalize the distribution. These SCR magnitudes were then averaged for each stimulus type separately by phase (acquisition, reversal, extinction) for each subject. Trials in which the CS coterminated with the US were analyzed separately.

The analysis of conditioning and extinction trials were guided by the results from the mouse. Specifically, we examined the data by run to test for changes in SCR magnitude over time. The humans, unlike the mouse, reached an asymptote in the SCR during extinction trials with no further decrease in SCR response for either genotype from extinction run 2 to run 3 (Val/Val: t(35)=−1.88, p<0.07; Val/Met: t(35)=−1.28, p<0.21). We therefore tested the effects of genotype for run 1 (early trials) and run 2 (late trials) with two separate tests with a Bonferroni correction for the two comparisons (0.05/2). The same analysis was applied for the fear conditioning trials to assess genotypic differences in response to the neutral cue over time. Similar to extinction, Val/Val homozygotes showed decrease in SCR response to the neutral cue from run 1 to run 2 (t(3S)=3.59, p<0.001, corrected for time), whereas Met allele carriers showed no change in response (t(3S)=0.94, p<0.37). Met allele carriers were slower to learn that the neutral cue represented safety as they did show a decrease in SCR from run 2 to run 3 (t(35)=3.61, P<0.001).

### Image Acquisition

Subjects were scanned with a 3.0 T General Electric Signa Excite HD MRI scanner (General Electric Medical Systems, Milwaukee, Wis.) of the Citigroup Biomedical Imaging Center at the Weill Cornell Medical College. A quadrature head coil was used to acquire all images. A whole brain, high resolution, Tl weighted anatomical scan (3D MPRAGE 256×256 in plane resolution, 240 mm FOV, 124×1.5 mm sagittal slices) was acquired for each subject for transformation and localization of functional data into Talairach space. Functional scans were T2*-weighted images acquired using a spiral in/out sequence (TR=2000, TE=30, FOV=200 mm, Flip angle=90° and 64×64 matrix) that covered the majority of the brain excluding the posterior portion of the occipital lobe. Each functional volume contained 29 5 mm thick coronal slices (skip 0) with an in-plane resolution of 3.125×3.125 mm.

### Imaging Data Analysis

Functional imaging data were preprocessed and analyzed using the Analysis of Functional Neurolmages (AFNI) software package. The first four volumes (8 s) from each of the nine runs were discarded to allow the scanner to reach magnetization equilibrium. Following slice time correction images were registered to the first functional volume using rigid body transformation. Head motion was examined to confirm that all subjects had less than 2 mm of translation or 2° of rotational movement. Trials with motion greater than 2 mm were discarded. The anatomical dataset was aligned to the first image volume of the functional dataset. Functional data were smoothed with an isotropic 6 mm Gaussian kernel. Time series were normalized to percent signal change to allow comparisons across runs and individuals by dividing signal intensity at each time point by the mean intensity for that voxel and multiplying the result by 100.

A general linear model (GLM) was performed for each participant to compute parameter estimates representing task effects at each voxel. Task regressors were created for each stimulus type (conditioned stimulus, neutral stimulus, unconditioned stimulus) specific to each phase (acquisition, reversal, extinction) by convolving the stimulus onset times with a gamma-variate hemodynamic response function. Linear and quadratic trends, as well as motion parameters, were modeled as regressors of non-interest to account for correlated drift and residual motion effects. Following GLM estimation, linear contrasts were computed to compare the parameter estimates representing task effects of interest, which were transformed into the standard coordinate space of Talairach and Tournoux. Talairached transformed images had are-sampled resolution of 3×3×3 mm. Normalization to Talairach space was performed using automatic Talairach transformation in AFNI, where the anatomical volume was warped using a 12-parameter affine transformation to a template volume (TT_N27) in Talairach space.

Group analyses focused on the vmPFC and the amygdala, two structures previously implicated in fear conditioning and extinction learning. Masks were generated to include only brain voxels within the boundaries of these anatomical structures bilaterally (vmPFC mask: ˜1000 cubic millimeters; amygdala: ˜890 cubic millimeters) as in prior work. The amygdala mask was created using boundaries provide by AFNI and the vmPFC mask was created by including all voxels between the Talairach coordinates of −1 0 and 10 in the x-plane, anterior to a in the y-plane and ventral to 14 in the z-plane. We performed Monte Carlo simulations on small volumes of the amygdala and ventromedial prefrontal cortex to generate the combination of p-value and cluster threshold that preserves alpha=0.05. For the amygdala, this was achieved by considering imaging results at p<0.05 with a 7 voxel minimum cluster size and for the vmPFC, this was achieved by considering imaging results at p<0.005 with a 7 voxel minimum cluster size. Application of these thresholds effectively preserved p<0.05, small-volume-corrected thresholding on all imaging results.

Within these masks, voxelwise random effects group analyses were performed to detect task and genotype effects. To identify effects of genotype during extinction, a between subjects t-test was performed to directly compare brain activity in Met and non-Met allele subjects to the conditioned stimulus, when it was no longer paired with the unconditioned stimulus, relative to resting fixation. The analysis directly parallels that used to test the main effect of genotype on SCR during extinction of the first conditioned stimulus with Met allele carriers showing less extinction ((F(1,70)=6.65, p<0.01); Val/Val: 0.29±0.02; Val/Met: 0.39±0.03). Results of the imaging analyses identified a single region within the vmPFC (x=−4, Y=24, z=3) and a single region within the left amygdala (x=−25, y=2, z=−20; see main text). The imaging results showed a dose response for 0, 1 or 2 Met alleles and the behavioral findings in SCR showed a less robust effect in SCR measure that may be due to the high variability in human behavioral measures. Parameter estimates were extracted from these regions and plotted by genotype for descriptive purposes. The only brain region outside of the vrnPFC and amygdala that exceeded a whole-brain threshold of p<0.05, corrected was a region near the posterior cingulate (x=1, y=−35, z=13; 10 voxels; t=−4.73; Val/Val>Val/Met).

### MRI-Based Morphometry

To examine whether the findings may be due to genotypic developmental effects on brain structure, we examined MRI-based morphometry. Specifically, parcellation of the subcortical anatomy into regions of interest (amygdala and hippocampus) and calculations of total brain volume were performed using the FreeSurfer software suite. An automated procedure was implemented which assigns a neuroanatomical label to each voxel in an MRI volume based on probabilistic information estimated from a manually labeled training set. The classification technique employs a non-linear registration procedure that is robust to anatomical variability. The segmentation uses three pieces of information to disambiguate labels: (1) the prior probability of a given tissue class occurring at a specific atlas location, (2) the likelihood of the image given what tissue class, and (3) the probability of the local spatial configuration of labels given the tissue class. The technique has previously been shown to be comparable in accuracy to manual labeling. The segmentations were visually inspected for accuracy by a single operator, and edited when necessary.

For analysis, relative volumes of the regions of interest, the hippocampus and amygdala, were calculated to take account of possible differences in brain volumes between subjects. This measure was obtained for each subject by dividing the area of interest volume (cm³) by that subject's total brain volume (cm³).

Overall total brain volume did not significantly differ between Met allele carriers (mean volume=1605 cm³, SD=145) and Val/Val homozygotes (mean volume=1581 cm³, SD=158; t(59)=−0.63, p<0.53). There was no significant difference between Val/Val and Met allele carriers volumetric measurements (mean adjusted volume represented as a percentage of total brain volume±SD) for either the amygdala [(t(59)=0.15, P<0.9); Val/Val: 0.10%±0.02; Val/Met: 0.10%±0.02] or the hippocampus [(t(59)=−1.95, P<0.06); Val/Val: 0.38%±0.06; Val/Met: 0.41%±0.05] although there was a trend for the hippocampal volume.

DTI and fMR1 Analysis of Frontolimbic Connectivity

Both human and mouse Met allele carriers showed impaired extinction learning, processes that are heavily dependent on the vmPFC and amygdala and the anatomical connectivity of this circuit. Responses in the vmPFC during extinction have been shown to correlate with the strength of amygdala activation in humans, consistent with the idea that the vmPFC is linked to diminished amygdala response. Hence, the observed impairment in extinction learning, which we found in both mice and human Met allele carriers, may be related to decreased fronto-amygdala connectivity. To test this idea, we quantified white matter connectivity in our human subjects using diffusion tensor imaging (DTI) by calculating the fractional anisotropy in the uncinate fasciculus (UF), the major white matter tract that connects the amygdala and the prefrontal cortex. Specifically, the rostral portion of the superior temporal gyrus and amygdala are connected to both the orbital and medial prefrontal cortex via the UF.

DTI studies were conducted on 82 subjects, 63 of whom had functional imaging data obtained on the fear conditioning and extinction task. DTI scans were obtained using a multislice, spin-echo, diffusion tensor pulse sequence (72 slices, 1.8 mm thick, TR=13500 msec, echo time=minimum, field of view 230 mm) covering the whole brain with one unweighted scan and diffusion-weighted scans in 55 independent directions.

Diffusion-weighted image reconstruction and DTI analysis were performed using the Analysis of Functional Neurolmages (AFNI) software package. As described in more detail elsewhere, anisotropic water diffusion can be modeled in terms of a 3×3 symmetric tensor (matrix). A pre-programmed AFNI algorithm was used to solve for the six independent components of this tensor in each voxel via transformations of the 55 non-collinear diffusion-weighted scans collected for each subject. Diagonalization (Jacobi transformation) of each voxel-specific tensor yielded three eigenvalues and three eigenvectors, respectively describing the magnitude and direction of water diffusion in each voxel, with the principal eigenvector representing motion in the direction of greatest diffusion. Fractional anisotropy (FA) was calculated in terms of these variables and approximates the degree to which water diffuses preferentially in one, principal direction (anisotropic) versus equally in all three directions (isotropic). In white matter, greater myelination and increased regularity in the orientation of axonal fibers is correlated with increased FA.

Using the procedure above, we generated fractional anisotropy maps quantifying the regularity and myelination of white matter throughout the whole brain. Next, each subject's scan was normalized to the standard coordinate space of Talairach and Tournoux using parameters obtained from the transformation of each subject's high-resolution anatomical scan. We then used a two-factor, mixed effects ANOVA (fixed: genotype, random: subjects) to compare FA in Val/Val versus Met allele carriers on a voxel-wise basis. White matter masks were made by averaging all subjects' FA maps and thresholding at a conservative value of FA>0.25 to restrict analysis to white matter voxels. Previous work has shown that this threshold reliably excludes the vast majority of gray matter voxels. We used a cluster correction for multiple comparisons within this white matter mask, with p<0.025 and N>99 voxels per cluster. The cluster threshold was selected using Monte Carlo simulation as implemented by AFNI's AlphaSim algorithm to obtain false positive rates of p<0.05. In regions of interest where between-group differences were detected, peak FA values were extracted and correlated with mean beta weights (percent signal change) from the vmPFC cluster that showed genotypic differences during extinction learning.

A voxel-based approach was used to investigate the association between frontolimbic white matter tracts and BDNF genotype. We found significant differences bilaterally in fractional anisotropy of the uncinate fasciculi between Val/Val and Met allele carriers (Right: F(1,82)=13.34, p<0.05 (corrected), Left: F(1,82)=12.46, p<0.05 (corrected) (potential FIG. 4A). Met allele carriers showed reduced fractional anisotropy in uncinate fasciculus tracts relative to homozygous Val carriers. The small number of homozygous Met allele carriers (n=3) precluded independent statistical analysis, and they were combined with Val/Met subjects. However, FA values from Met/Met subjects were plotted separately to see the dose response of the allele, (potential FIG. 4B) showing that FA in the UF in the homozygote Met/Met, like Val/Met subjects were significantly different from the Val/Val group.

We then tested to what extent the strength of frontolimbic fiber tracts was associated with both our behavioral (SCR) and functional (BOLD) measures of extinction Skin conductance response to the conditioned stimulus during extinction did not correlate with fractional anisotropy values in the uncinate fasciculus (r=0.04, P<0.73), but genotypic differences in functional activity in the vmPFC during extinction were correlated with fractional anisotropy measures in the left uncinate fasciculus (potential FIG. 5). In this analysis, we specifically focused on the left uncinate fasciculus tract since we found that during extinction, the observed genotypic-dependent difference in amygdala activity was localized to the left side.

To fully characterize the pattern of responding 111 the vmPFC, mean BOLD responses to extinction stimuli were extracted for all active vmPFC voxels (189 mm³). We looked at brain activity in this region to previously conditioned stimulus during extinction and found genotypic differences in the vmPFC during extinction (t(68)=−3.78, p<0.05) to the conditioned cue. Measures of BOLD signal representing brain activation in this region (beta weights) were then extracted from this region of the vmPFC for each subject as described above and correlated with the peak FA value from the UF. We found FA significantly correlated with the functional activity during the extinction phase (r=0.26, p<0.04). That is, higher FA in white matter tracts connecting the amygdala and prefrontal cortex was associated with greater vmPFC recruitment during extinction. This effect was predominantly driven by Val/Val carriers (r=0.47, p<0.01) and not Met allele carriers (r=−0.241, p<0.170) during the extinction phase.

We found significant genotypic differences in fractional anisotropy in the uncinate fasciculus, with Met allele carriers having lower connectivity in this frontolimbic tract. Critically, this was not a result of simple volume differences between Val/Val and Met allele carriers, since the BDNF polymorphism did not influence total brain volume, or relative amygdala volume within our sample. Moreover, fractional anisotropy in the uncinate fasciculus in Val/Val homozygotes was correlated with vmPFC functional activity during extinction, consistent with the idea that greater connectivity between the amygdala and vmPFC results in better vmPFC suppression of the amygdala, and hence more effective extinction learning.

Impaired extinction learning has been associated with healthy human Met allele carriers, and is similarly characteristic of patients with anxiety disorders. The Val66Met BDNF polymorphism has been shown to increase risk for anxiety disorders in humans and anxious behavior in the mouse. Our finding of decreased connectivity in frontolimbic tracts in Met allele carriers as well as an anxious phenotype in the mouse model is consistent with findings of lower FA values in the uncinate fasciculus in patients with anxiety disorders relative to controls and recent work by others showing that the strength of axonal pathway connecting amygdala and prefrontal regions is inversely correlated with trait anxiety.

Not being bound by theory, the inventors believe that the above findings are most likely due to the SNP biasing activity-dependent learning rather than affecting CNS development per se as there was no evidence of genotypic developmental effects on brain structure in this ethnicity-, age- and gender-matched sample using MR1-based brain morphometry. Furthermore, an association between vmPFC activity and strength of fibers connecting frontolimbic regions is consistent with more effective extinction learning as a result of better vmPFC modulation of the amygdale.

1. A method for identifying an increased risk for anxiety disorders in a human, the method comprising testing said human for the presence of brain derived neurotrophic factor (BDNF) Val-66-Met genotype, and identifying an increased risk for anxiety disorders in humans with the BDNF Val-66-Met genotype.

2. The method according to claim 1, wherein the anxiety disorder is post traumatic stress disorder.

3. The method according to claim 1, wherein the anxiety disorder is a phobia.

4. The method according to claim 1, further comprising administering a fear conditioning procedure to the human while measuring fear, and administering an extinction procedure to the human while measuring fear, wherein a prolonged time period during extinction in the human identifies an increased risk for anxiety disorders.

5. A method for identifying risk for anxiety disorders in a human, the method comprising comparing fMRI images of the amygdala of the human acquired during conditioning and extinction therapy, determining if the human is unresponsive to extinction therapy by noting heightened and non-declining activity in the amygdala during extinction, and indentifying risk for anxiety if there is heightened and non-declining activity in the amygdala during extinction.

6. The method according to claim 5, wherein the anxiety disorder is post traumatic stress disorder.

7. The method according to claim 5, wherein the anxiety disorder is a phobia.

8. A method for identifying a human that will be non-responsive to behavioral treatments for anxiety disorders, the method comprising testing said human for the presence of brain derived neurotrophic factor (BDNF) Val-66-Met genotype, and identifying the human to be non-responsive to behavioral treatments for anxiety disorders.

9. The method according to claim 8, wherein the anxiety disorder is post traumatic stress disorder.

10. The method according to claim 8, wherein the anxiety disorder is a phobia.
